(19) United States
(12) Patent Application Publication
(10) Pub. No.: US 2021/0236849 A1
(43) Pub. Date: Aug. 5, 2021

(54) METHOD FOR PREPARING FOR BRACHYTHERAPY, BRACHYTHERAPY METHOD, AND BRACHYTHERAPY SYSTEM

(71) Applicant: Carl Zeiss Meditec AG, Jena (DE)

(72) Inventors: Valentin Neis, Ulm (DE); Friederike Neis, Ulm (DE); Frank Weigand, Heidenheim (DE)

(21) Appl. No.: 17/163,280

(22) Filed: Jan. 29, 2021

(30) Foreign Application Priority Data
Jan. 30, 2020 (DE) 10 2020 102 213.1

(51) Int. Cl. A61N 5/10; G16H 20/40

(52) U.S. Cl. CPC A61N 5/1007; A61B 34/20; A61N 2005/1074; G16H 20/40

(57) ABSTRACT

A method for preparing for brachytherapy, a brachytherapy method, and a brachytherapy system are provided and involve using an applicator to be placed onto a radiation source, and an applicator guide to be inserted into an object to be irradiated. The applicator guide being formed such that the applicator can be inserted into the applicator guide. A position and orientation of the applicator guide are sensed by a navigation system, a position and orientation of an insertion assistance graphic object in a graphical representation are determined based on the sensed position and orientation of the applicator guide by a data processing device and the insertion assistance graphic object is displayed in the graphical representation by a display device. By its shape and being presented in the graphical representation, the insertion assistance graphic object is suitable for making it easier for a viewer to insert the applicator into the applicator guide.

### CROSS REFERENCE TO RELATED APPLICATIONS

This application claims priority to German patent application DE 10 2020 102 213.1, filed Jan. 30, 2020, the entire content of which is incorporated herein by reference.

### TECHNICAL FIELD

The present disclosure relates to a method for preparing for brachytherapy, to a brachytherapy method, and to a brachytherapy system. In particular, the present disclosure relates to an aid for inserting an applicator, which can be placed onto a radiation source, into an applicator guide, which can be inserted into an object to be irradiated.

### BACKGROUND

In the area of brachytherapy using x-radiation, there are therapies in which it is intended for x-radiation to be generated inside an object, in particular a human or animal body, in the vicinity of the region to be irradiated, in particular in the vicinity of tissue to be treated. The x-radiation may be provided here by an irradiation unit. An exemplary irradiation unit includes a particle beam system, which can generate a high-energy particle beam. The particle beam is directed through a tube of the irradiation unit, which is several centimeters long, onto an x-ray material disposed at the end of the tube. Interaction of the particle beam with the x-ray material causes x-radiation to be emitted from the x-ray material. This x-radiation emerges from the tube and impinges on the region to be irradiated.

In order that the x-radiation generated by the x-ray material at the tip of the tube can be applied inside an object, the tube is inserted into the object. For this purpose, the tube is surrounded by an applicator, which on the one hand provides a sterile barrier and on the other hand protects the tube.

Before the insertion of the tube into the object, an applicator guide is inserted into the object. The applicator guide includes for example a tubular portion, which has at one end an opening for the insertion of the applicator. Once the applicator guide has been inserted into the object, the irradiation unit provided with the applicator can be inserted into the applicator guide through the opening of the tubular portion. In this way, the x-ray material is disposed inside the object. Then, the x-ray material can generate the x-radiation inside the object by interacting with the particle beam.

With this procedure, there is however the problem that, as soon as the applicator guide has been inserted into the object, only a short portion of the applicator guide can still be seen. It can therefore be difficult to ascertain the orientation of the applicator guide by eye. When the applicator is inserted into the applicator guide, the applicator must however be aligned relatively accurately in relation to the applicator guide, in order that the applicator and the tube of the irradiation unit disposed therein are not bent during insertion. If the tube is bent, generation of the x-radiation is disrupted.

### SUMMARY

It is therefore an object of the present disclosure to provide an aid that makes it easier for the applicator to be inserted into the applicator guide.

A first aspect of the disclosure concerns a method for preparing for brachytherapy, the method comprising: providing an applicator and an applicator guide, wherein the applicator guide is formed such that the applicator can be inserted into the applicator guide and the applicator guide guides the movement of the applicator during the insertion of the applicator into the applicator guide, sensing a position and orientation of the applicator guide by a navigation system, determining a position and orientation of an insertion assistance graphic object in a graphical representation on the basis of the sensed position and orientation of the applicator guide by a data processing device, displaying the insertion assistance graphic object in the graphical representation at the determined position in the determined orientation by a display device, wherein the insertion assistance graphic object is suitable by virtue of its shape for making it easier for a viewer to insert the applicator into the applicator guide.

The position and orientation of the insertion assistance graphic object can be determined for example by calculation by the data processing device, the calculation using the sensed position and orientation of the applicator guide. Since the position and orientation of the insertion assistance graphic object are determined in dependence on the sensed position of the (real-world) applicator guide, the position and orientation of the insertion assistance graphic object change when the position and orientation of the (real-world) applicator guide change.

A graphic object refers here to a virtual object that is only made visible to a human observer by its visual representation. A graphic object has no body of any mass. A graphic object has a shape that can replicate a shape of a real-world object. For example, the insertion assistance graphic object may have a shape that replicates the shape of the applicator. The shape of a real-world object is provided by its physical form.

Individual steps or all steps of the procedure may be repeated. For example, the sensing of the position and orientation of the applicator guide, the determining of the position and orientation of the insertion assistance graphic object in the graphical representation and the displaying of the insertion assistance graphic object may be repeated. As a result, a change of the position and orientation of the applicator guide has an effect on the representation of the insertion assistance graphic object to the extent that the insertion assistance graphic object is presented at the newly determined position in the newly determined orientation.

The above procedure brings about the effect that the insertion assistance graphic object is presented at a determined position in a determined orientation, and consequently can be visually viewed by a viewer wishing to insert the applicator into the applicator guide. By virtue of its shape and its determined position and orientation, the insertion assistance graphic object is an aid to the viewer when inserting the applicator into the applicator guide, since it provides the viewer with a guide or reference for moving the applicator.

A second aspect of the disclosure concerns a brachytherapy method. The brachytherapy method includes the steps of the method for preparing for brachytherapy according to the first aspect, wherein, before the sensing of the position and orientation of the applicator guide, the applicator guide is inserted into an object, in particular a human or animal body, and wherein, during the sensing of the position and orientation of the applicator guide, the position and orientation of the applicator guide inserted into the object are sensed. The brachytherapy method may in particular also include inserting the applicator into the applicator guide by using the presented insertion assistance graphic object, and/or generating x-radiation inside the applicator inserted into the applicator guide.

A third aspect of the disclosure concerns a system, in particular a brachytherapy system, which is configured such that it allows the methods described herein to be carried out. The system includes in particular an applicator, an applicator guide, a navigation system, a data processing device and a display device.

### BRIEF DESCRIPTION OF THE DRAWINGS

The disclosure will now be described with reference to the drawings wherein:

FIG. 1 shows a schematic representation of an irradiator, an applicator, and an applicator guide;

FIG. 2 shows a schematic representation of a brachytherapy system;

FIG. 3 shows a schematic representation of a graphical representation with objects of enhanced reality for being displayed by a display device;

FIG. 4 shows a schematic representation of a further graphical representation with objects of enhanced reality for being displayed by a display device;

FIG. 5 shows a schematic representation of a further graphical representation with objects of enhanced reality for being displayed by a display device; and

FIG. 6 shows a schematic representation of a graphical representation with objects of virtual reality for being displayed by a display device.

### DESCRIPTION OF EXEMPLARY EMBODIMENTS

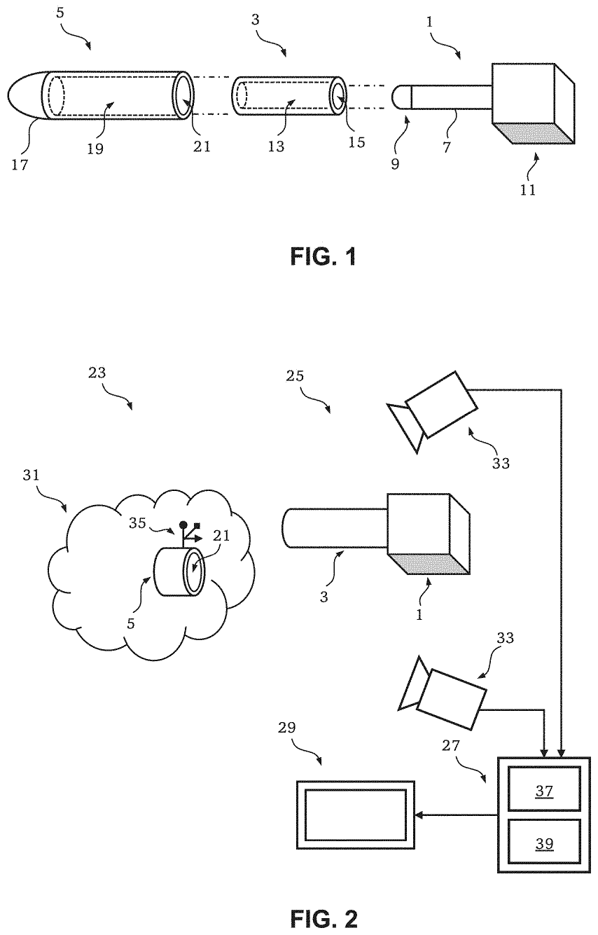

FIG. 1 shows in a schematic representation an irradiator or irradiation unit 1, an applicator 3 and an applicator guide 5.

The irradiator 1 includes a tube 7, which is several centimeters long, at one end of which an x-ray material 9 is arranged. The irradiator 1 also includes a particle beam system 11, which is configured to generate a particle beam and to direct it through the tube 7 onto the x-ray material 9. As a result, x-radiation is generated by interaction of the particle beam with the x-ray material 9. The irradiator 1 may be carried by a stand, which makes it easier for a user to handle (move and orient) the irradiator 1. The irradiator 1 may be differently configured. The disclosure can be applied to all irradiators that have the tube 7 or a functionally similar elongated portion.

In the exemplary embodiment shown in FIG. 1, the applicator 3 has a cylindrical shape. However, the applicator 3 may also have different shapes. The applicator 3 has a cavity 13 inside the applicator 3 that is adapted to the shape of the tube 7 of the irradiator 1. The cavity 13 is schematically represented by dashed lines. The cavity 13 extends up to an opening 15 of the applicator 3. The opening 15 and the cavity 13 adjoining it are shaped such that the tube 7 of the irradiator 1 can be at least partially inserted through the opening 15 into the cavity 13 of the applicator 3. The tube 7 can in practice be inserted several centimeters into the cavity 13. The shape of the cavity 13 of the applicator 3 brings about the effect of guiding the tube 7 during the insertion of the tube 7 into the cavity 13 of the applicator 3. If the applicator 3 is in this way placed onto the irradiator 1, the applicator 3 protects the tube 7 of the irradiator 1 from external influences.

In the example shown in FIG. 1, the applicator guide 5 has a substantially cylindrical shape with a rounded end 17. The rounded end 17 serves for easy insertion of the applicator guide 5 into an object. The applicator guide 5 has a cavity 19 inside it, which extends up to an opening 21. The opening 21 and the cavity 19 of the applicator guide 5 are shaped such that the applicator 3 can be at least partially inserted through the opening 21 into the cavity 19 of the applicator guide 5. The applicator guide 5 can in practice be inserted several centimeters into the object 31. The shape of the cavity 19 of the applicator guide 5 brings about the effect of guiding the applicator 3 during the insertion of the applicator 3 into the cavity 19 of the applicator guide 5.

FIG. 2 shows in a schematic representation of a brachytherapy system 23 with which the procedures described herein can be carried out. The brachytherapy system 23 includes the applicator 3, the applicator guide 5, a navigation system 25, a data processing device 27 and a display device 29.

In the situation shown in FIG. 2, the irradiator 1 has been inserted into the applicator 3. Furthermore, the applicator guide 5 has been inserted into an object 31. Only a short portion of the applicator guide 5, which has the opening 21, still protrudes out of the object 31. This is a typical situation during a brachytherapy procedure. This clearly demonstrates the problem that the orientation of the applicator guide 5 is difficult to determine, since a large part of the applicator guide 5 is arranged in the object 31 and is therefore not visible. It is therefore difficult to insert the applicator 3 into the applicator guide 5, since the orientation of the applicator 3 has to be aligned with the orientation of the applicator guide 5, which however is difficult when the orientation of the applicator guide 5 itself is difficult to determine.

This problem is solved by the brachytherapy system 23 as explained below.

The navigation system 25 is configured for sensing the position and the orientation of the applicator guide 5. The navigation system 25 may be configured in various ways. The navigation system 25 represented in the figures and described herein merely shows one possible configuration.

The navigation system 25 includes a number of cameras 33, which are configured to record an image or a video of the applicator guide 5 (inserted into the object 31). The cameras 33 may be for example conventional color image cameras.

The navigation system 25 also includes a marking 35. The marking 35 can be connected to the applicator guide 5 in a known position and orientation with respect to the applicator guide 5. When the marking 35 is connected to the applicator guide 5 in the known position and orientation with respect to the applicator guide 5, as is shown in FIG. 2, the orientation of the marking 35 changes according to the change of the orientation of the applicator guide 5. When the marking 35 is connected to the applicator guide 5 in the known position and orientation with respect to the applicator guide 5, the position and orientation of the applicator guide 5 can be determined on the basis of the position and orientation of the marking 35, since the spatial interrelationship is known.

In the exemplary embodiment shown in FIG. 2, the marking 35 consists of three rods which are respectively oriented orthogonally in relation to one another. At the ends of the rods are a ball, a cube, and a pyramid. This configuration of the marking 35 allows the position and orientation of the marking 35 to be determined on the basis of the images that are recorded by the cameras 33. The configuration of the marking 35 explained in conjunction with FIG. 2 is merely given by way of example. For example, instead of the ball, the cube and the pyramid, other distinguishable marking elements may be used, for example three identical symbols of different colors or the like.

The navigation system 25 also includes a data processing portion 37, which is for example provided in the data processing device 27. The data processing portion 37 for the navigation system 25 receives the images recorded by the cameras 33 and determines from them the position and orientation of the marking 35 and from them the position and orientation of the applicator guide 5. The determination of the position and orientation of the applicator guide 5 is not restricted to the example described here. Other techniques may be used for this.

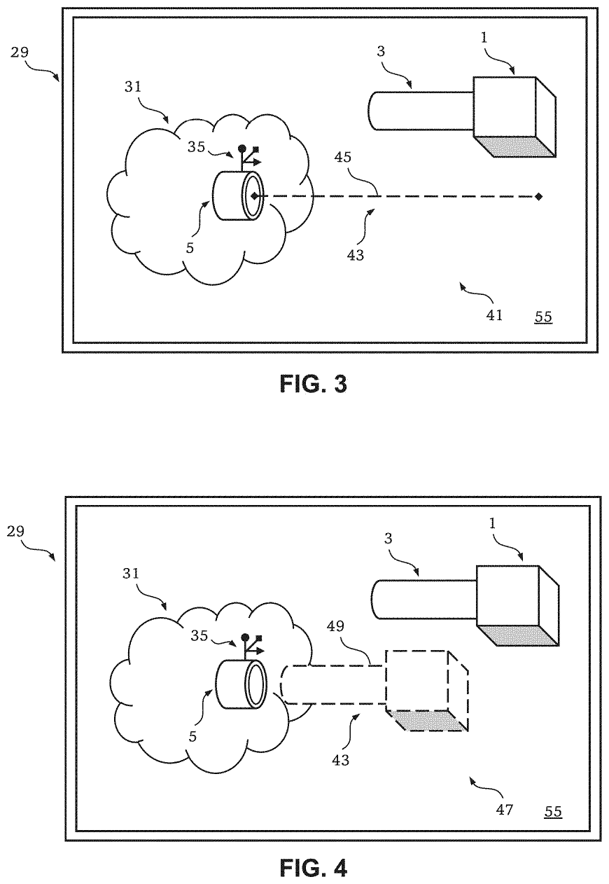

On the basis of the sensed position and orientation of the applicator guide 5, a data processing portion 39 of the data processing device 27 determines a position and orientation of an insertion assistance graphic object in a graphical representation, which is displayed with the display device 29. This is explained in more detail below with reference to FIGS. 3 and 4.

FIG. 3 shows a schematic representation of a graphical representation 41, which is generated by the data processing device 27 and is displayed in a visually perceptible manner by the display device 29. The graphical representation 41 may include a plurality of graphic objects.

The display device 29 is for example a pair of “augmented reality” glasses (AR glasses), which enables a user to simultaneously view on the one hand a real-world scene directly and on the other hand virtual objects of augmented reality. The display device 27 similar to AR glasses has a display area 55, which can be viewed by a viewer. The graphical representation 41 is displayed in a visually perceptible manner on the display area 55. The display area 55 is otherwise transparent, so that the real-world scene can be viewed directly in the areas of the display area 55 in which the graphical representation 41 does not have any graphic objects. The graphic objects contained in the graphical representation 41 can therefore be viewed in the context of the real-world scene. In this way, the AR glasses augment the real-world scene with graphic objects of the graphical representation 41 that are not contained in the real-world scene itself.

FIG. 3 shows in a schematic representation the graphical representation 41 in the context of a real-world scene through a display device 27 similar to AR glasses.

The real-world scene includes the irradiator 1, the applicator 3, the applicator guide 5 with marking 35, and the object 31.

The graphical representation 41 includes an insertion assistance graphic object 43. In the exemplary embodiment shown in FIG. 3, the insertion assistance graphic object 43 includes a guideline 45. The guideline 45 is represented by a dashed line with rhomboids at the ends. The graphical representation of guidelines is not restricted to this. The display device 29 showing the graphical representation 41 with the guideline 45 in the context of the real-world scene makes it easier for a viewer to insert the applicator 3 into the applicator guide 5.

The position and orientation of the insertion assistance graphic object 43 or the position and orientation of the guideline 45 in the graphical representation 41 are determined by the data processing portion 39 for example such that the insertion assistance graphic object 43 or the guideline 45 appears as an extension of the cavity 19 of the applicator guide 5. This means for example that the guideline 45 is displayed as though it were oriented parallel to the cylindrical cavity 19 of the applicator guide 5 in the real-world space. The guideline 45 may in this case be positioned in the graphical representation 41 such that it appears as though the guideline 45 passes through the opening 21 of the applicator guide 5.

In order to be able to align the position and orientation of the insertion assistance graphic object 43 or the guideline 45 in the graphical representation 41 with the real-world position and orientation of the applicator guide 5, the data processing portion 39 determines the position and orientation of the insertion assistance graphic object 43 or the guideline 45 on the basis of the position and orientation of the applicator guide 5 previously sensed with the application system 25.

As it is/they are displayed by the AR glasses, the graphical representation or the position and orientation of the graphic objects of the graphical representation depend(s) on the position and direction from which the viewer views the real-world scene. To be able to take this into account, the navigation system 25 may also be configured to sense the position and orientation of the display device 29, and the data processing device 27 may be configured to determine the position and orientation of the insertion assistance graphic object 43 in the graphical representation 41 also on the basis of the sensed position and orientation of the display device 29.

The data processing device 27 causes the display device 29 to display the insertion assistance graphic object 43 at the determined position in the determined orientation in the graphical representation 41. By virtue of its shape and being presented at the determined position in the determined orientation, the insertion assistance graphic object 43 is suitable for making it easier for a viewer to insert the applicator 3 into the applicator guide 5. This takes place by the viewer viewing the displayed graphical representation 41 in the context of the real-world scene and, by virtue of the shape and the position and orientation of the presented insertion assistance graphic object 43, the viewer being provided with a visual aid for making the positioning and orientation of the applicator 3 easier.

FIG. 4 shows in a schematic representation a further graphical representation 47 in the context of a real-world scene by a display device 27 similar to AR glasses. The graphical representation 47 is generated by the data processing device 27 and displayed by the display device 29.

In the graphical representation 47 shown in FIG. 4, the insertion assistance graphic object 43 includes a graphic object 49, which reproduces the shape of the applicator 3 (and of the irradiation unit 1). The data processing portion 39 determines the position and orientation of the graphic object 49 in the graphical representation 47 for example such that they represent a target position and a target orientation for the applicator 3 that are to be achieved by moving the applicator 3 in order to be able to insert the applicator 3 into the applicator guide 5. For example, the position and orientation of the graphic object 49 in the graphical representation 47 are determined such that the graphic object 49 appears as an extension of the cavity 19 of the applicator guide 5. To present the graphic object 49 in the shape of the applicator 3, the data processing device 27 includes a data memory, from which form data that represent the shape of the applicator 3 can be obtained. Form data represent the spatial shape of a real-world object. The form data are used for presenting the graphic object 49 or for presenting the insertion assistance graphic object 43.

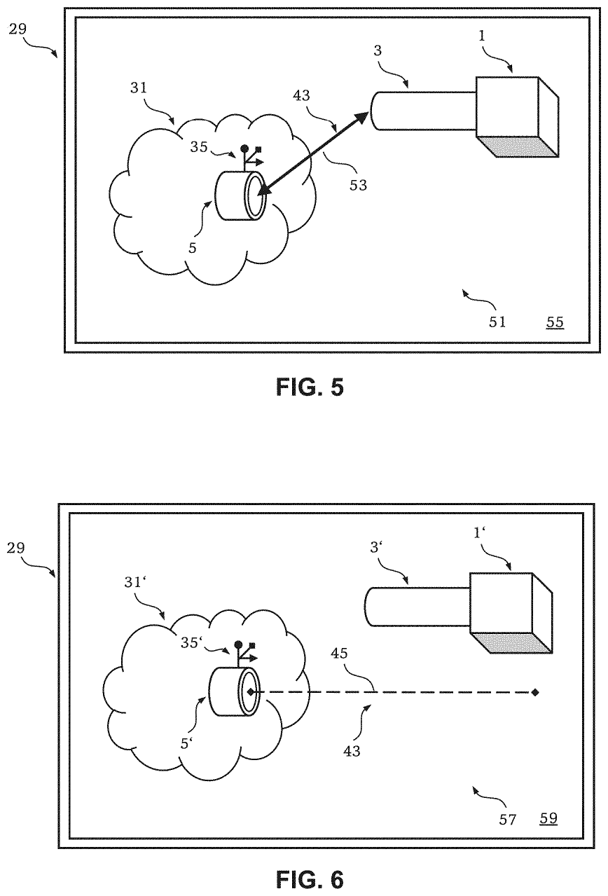

A further exemplary embodiment is now described with reference to FIG. 5. The navigation system 25 may also be configured to determine the position and/or orientation of the applicator 3. The technique for sensing the position and/or orientation of the applicator 3 may be the same as the technique for sensing the position and orientation of the applicator guide 5. Alternatively, the position and/or orientation of the applicator 3 may be sensed by the navigation system 25 by a different technique.

The data processing portion 39 may also be configured to determine an auxiliary variable on the basis of the sensed position and/or orientation of the applicator 3 and the sensed position and/or orientation of the applicator guide 5. The auxiliary variable is for example a distance between the applicator 3 and the applicator guide 5. For determining the distance between the applicator 3 and the applicator guide 5, the data processing portion 39 uses for example the position of the applicator 3 and position of the applicator guide 5 sensed with the navigation system 25 and also form data that represent the shape of the applicator 3 and the shape of the applicator guide 5. Depending on the type of auxiliary variable, the determining of the auxiliary variable is based on the sensed position of the applicator 3 and/or the sensed orientation of the applicator 3 and/or the sensed position of the applicator guide 5 and/or the sensed orientation of the applicator guide 5 and/or form data concerning the applicator 3 and/or form data concerning the applicator guide 5.

The data processing portion 39 is also configured to present the auxiliary variable as part of the insertion assistance graphic object 43.

FIG. 5 shows a schematic representation of a graphical representation 51 in the context of a real-world scene by a display device 27 similar to AR glasses.

The graphical representation 51 is generated by the data processing device 27 and displayed by the display device 29. The graphical representation 51 includes an insertion assistance graphic object 43, which includes an auxiliary variable 53. The auxiliary variable 53 is depicted by a double-headed arrow and represents the distance determined by the data processing portion 39 between the applicator 3 and the applicator guide 5.

Examples of graphical representations that are displayed by a display device 27 similar to AR glasses in the context of a real-world scene have been explained with reference to FIGS. 3 to 5. The type of display is not restricted to these. An exemplary embodiment in which the insertion assistance graphic object 43 is presented as an object of virtual reality or in an image is explained below with reference to FIG. 6.

FIG. 6 shows a schematic representation of a graphical representation 57, which is generated by the data processing device 27 and is displayed by the display device 29. The display device 29 is for example a pair of “virtual reality” glasses or a monitor. These devices display to a viewer an image 59, which includes the graphical representation 57. The image area of the image 59 does not include any real-world objects, but exclusively objects of virtual reality. Conventional photography is like this, that is to say it records a real-world scene and reproduces the recording in a form that can be seen visually, an image, which exclusively presents objects of virtual reality.

The graphical representation 57 includes a plurality of graphic objects, which represent real-world objects. For example, the graphical representation 57 includes an image area 1′, which represents the reproduction of the recording of the irradiation unit 1, an image area 3′, which represents the reproduction of the recording of the applicator 3, an image area 5′, which represents the reproduction of the recording of the applicator guide 5, and an image area 31′, which represents the reproduction of a recording of the object 31. The image areas 1′, 3′, 5′, and 31′ are graphic objects of the graphical representation 57 that represent real-world objects.

The graphical representation 57 also includes graphic objects that represent virtual objects. The graphical representation 57 includes for example the insertion assistance graphic object 43, which has been added to the recording of the real-world scene by image processing by the data processing portion 39.

By virtue of its shape and being presented at the determined position in the determined orientation, the insertion assistance graphic object 43 is suitable for making it easier for a viewer to insert the applicator 3 into the applicator guide 5. This takes place by the viewer viewing the displayed graphical representation 57 and, by virtue of the shape and the position and orientation of the presented insertion assistance graphic object 43, the viewer being provided with a visual aid for making the positioning and orientation of the applicator 3 easier.

It is understood that the foregoing description is that of the exemplary embodiments of the disclosure and that various changes and modifications may be made thereto without departing from the spirit and scope of the disclosure as defined in the appended claims.

What is claimed is:

1. A method for preparing for brachytherapy, the method comprising:
providing an applicator and an applicator guide, the applicator guide being formed such that the applicator can be inserted into the applicator guide, and the applicator guide guides a movement of the applicator during insertion of the applicator into the applicator guide;
sensing a position and an orientation of the applicator guide by a navigation system;
determining, by a data processing device, the position and the orientation of an insertion assistance graphic object in a graphical representation based on the position and the orientation of the applicator guide sensed by the navigation system;
displaying the insertion assistance graphic object at the position and the orientation, determined by the data processing device, in the graphical representation by a display device; and
the insertion assistance graphic object being suitable by virtue of a shape of the insertion assistance graphic object for making it easier for a viewer to insert the applicator into the applicator guide.

2. The method according to claim 1, wherein the insertion assistance graphic object comprises a graphic object which reproduces the shape of the applicator.

3. The method according to claim 2, further comprising:
obtaining form data, which represent the shape of the applicator, from a data memory; and
displaying the insertion assistance graphic object based on the form data.

4. The method according to claim 1, wherein the insertion assistance graphic object comprises at least one guideline.

5. The method according to claim 1, wherein the position and the orientation of the insertion assistance graphic object are aligned with the position and the orientation of the applicator guide sensed by the navigation system.

6. The method according to claim 1, wherein the applicator guide comprises a cavity, into which the applicator is to be inserted, and
wherein the position and the orientation of the insertion assistance graphic object in the graphical representation are determined such that the insertion assistance graphic object appears as an extension of the cavity.

7. The method according to claim 1, wherein the position and the orientation of the insertion assistance graphic object are determined such that the position and the orientation of the insertion assistance graphic object represent a target position and a target orientation for the applicator that are to be achieved by moving the applicator such that the applicator can be inserted into the applicator guide.

8. The method according to claim 1, further comprising:
sensing the position and the orientation of the applicator by the navigation system;
determining an auxiliary variable, in particular a distance between the applicator and the applicator guide, based on the position and/or the orientation of the applicator sensed by the navigation system and the position and/or the orientation of the applicator guide sensed by the navigation system; and
displaying the auxiliary variable as part of the insertion assistance graphic object.

9. The method according to claim 1, further comprising:
sensing the position and the orientation of the applicator by the navigation system;
determining a distance between the applicator and the applicator guide based on the position and/or the orientation of the applicator sensed by the navigation system and the position and/or the orientation of the applicator guide sensed by the navigation system; and
displaying the distance between the applicator and the applicator guide as part of the insertion assistance graphic object.

10. The method according to claim 1, further comprising:
sensing the position and the orientation of the display device; and
determining the position and the orientation of the insertion assistance graphic object in the graphical representation based on the position and the orientation of the display device.

11. The method according to claim 1, wherein the insertion assistance graphic object is presented as at least one of (i) an object of enhanced reality, (ii) an object of virtual reality, and (iii) in an image.

12. A brachytherapy method, comprising:
the method according to claim 1;
inserting the applicator guide into an object before sensing the position and orientation of the applicator guide, and
during sensing the position and orientation of the applicator guide, sensing the position and orientation of the applicator guide inserted into the object.

13. The brachytherapy method according to claim 12, wherein the object is a human body or an animal body.

14. The brachytherapy method according to claim 12, further comprising at least one of:
inserting the applicator into the applicator guide by using the insertion assistance graphic object displayed by the display device; and
generating x-radiation inside the applicator inserted into the applicator guide.

15. A brachytherapy system, comprising:
the applicator;
the applicator guide;
the navigation system;
the data processing device; and
the display device,
wherein the brachytherapy system is configured to carry out the method according to claim 1.
